(19) United States
(12) Patent Application Publication
(10) Pub. No.: US 2025/0134470 A1
(43) Pub. Date: May 1, 2025

(54) COMPUTER-IMPLEMENTED METHOD FOR CLASSIFYING PHYSIOLOGICAL SIGNALS AND USE FOR MEASURING THE SPECIFIC MUSCULAR ACTIVITY OF A MUSCLE GROUP OF A SUBJECT

(71) Applicant: BLUEBACK, CESSON SEVIGNE (FR)

(72) Inventors: Caroline PAUTARD, PARTHENAY DE BRETAGNE (FR); Clément JOUANNEAU, PARTHENAY DE BRETAGNE (FR); Inès MOUDJARI, LA GAUDE (FR)

(21) Appl. No.: 18/837,883

(22) PCT Filed: Feb. 20, 2023
(86) PCT No.: PCT/EP2023/054139

§ 371 Date: Aug. 12, 2024

(30) Foreign Application Priority Data
Feb. 21, 2022 (EP) 22305187.1
Sep. 8, 2022 (EP) 22306332.2

(51) Int. Cl. A61B 5/00; A61B 5/296; A61B 5/397

(52) U.S. Cl. CPC A61B 5/7267; A61B 5/296; A61B 5/397; A61B 5/7207

(57) ABSTRACT

The present invention relates to a computer-implemented method for classifying physiological signals arising from the specific muscular activity of a muscle group comprising at least one deep muscle and at least one superficial muscle of a subject. The present invention also relates to a method for measuring the specific muscular activity of a muscle group comprising at least one deep muscle and at least one superficial muscle of a subject. The present invention further relates to the measuring method being used for an application chosen from among functional rehabilitation, muscle strengthening for wellbeing and/or aesthetic reasons, prevention and functional diagnosis, as well as to a computer program product.

### CROSS-REFERENCE TO RELATED APPLICATIONS

The present application is a filing under 35 U.S.C. 371 as the National Stage of International Application No. PCT/EP2023/054139, filed Feb. 20, 2023, entitled “COMPUTER-IMPLEMENTED METHOD FOR CLASSIFYING PHYSIOLOGICAL SIGNALS AND USE FOR MEASURING THE SPECIFIC MUSCULAR ACTIVITY OF A MUSCLE GROUP OF A SUBJECT,” which claims priority to European Application No. 22305187.1 filed with the European Patent Office on Feb. 21, 2022, which also claims priority to European Application No. 22306332.2 filed with the European Patent Office on Sep. 8, 2022, all of which are incorporated herein by reference in their entirety for all purposes.

### DESCRIPTION

### Technical Field

The present invention relates to a computer-implemented method for classifying physiological signals arising from the specific muscular activity of a muscle group comprising at least one deep muscle and at least one superficial muscle of a subject, as well as to a method for measuring the specific muscular activity of a muscle group comprising at least one deep muscle and at least one superficial muscle of a subject, to the use of said method and to a computer program product.

The present invention has applications in particular in the fields of functional rehabilitation, sports, well-being, aesthetics and functional diagnosis.

In the following description, references enclosed in brackets ([ ]) refer to the list of references presented at the end of the text.

### State of the Art

In the field of non-invasive measurement of muscular activity, it is known to use electromyography (EMG), which involves positioning adhesive electrodes on the patient's skin strategically in relation to the anatomy of the muscle for which muscular activity is to be recorded.

This technique is limited by the fact that the signal collected can only be specific to the targeted muscle if said muscle is positioned just below the measuring electrode. It therefore mainly applies to the so-called superficial muscles, located just below the skin.

The SENIAM project (http://www.seniam.org/) was conducted with the aim of consolidating applied research in the field of surface electromyography, resulting in the publication of European recommendations concerning sensors, optimal positioning thereof by targeted muscle, and suitable signal processing methods. These recommendations therefore concern 30 individual muscles, but do not include deep muscles. Similarly, there is a dichotomy between the number of sensors and the number of muscles to be characterized: in order to know the specific muscular activity of a muscle, at least one sensor is needed. The same sensor cannot be used to specifically characterize more than one muscle. If a sensor is used to characterize more than one superficial muscle, the information gathered is therefore combined, that is, we know that one of the muscles is contracted, or that all the muscles are relaxed, but no additional information can be deduced.

At the same time, there have been several attempts to measure deep muscle activity using surface EMG electrodes.

McGill et al. (1996, J. Biomechanics, Vol. 29, No. 11, pp. 1503-1507 ([1])) studied the possibility of measuring the muscular activity of 5 specific muscles (psoas, external oblique, internal oblique, transverse abdominis and quadratus lumborum) using a single surface EMG electrode strategically positioned for each muscle. This study shows that certain surface anatomical sites could be used to reflect the muscular activity of deep muscles such as the quadratus lumborum and the psoas. However, the measurement error is 6% to 12%. In addition, the findings show that the location which would allow measurement (with an error of 15%) of the transverse abdominis is the same as that which best represents the muscular activity of the internal oblique (15 cm lateral to the navel, just above the inguinal ligament). As a result, this localization is not specific to one of these two muscles, and the method thus disclosed does not make it possible to measure the specific muscular activity of a group of muscles comprising at least one deep muscle and at least one superficial muscle.

Jesinger and Stonick (1994, 6th IEEE DSP Workshop Proc., pp. 57-60 ([2])) propose to solve an inverse problem based on finite element modeling of deep muscle electrical activity and its propagation to the skin surface. This method, like others based on similar principles of model-based inverse solving, requires a large number of electrodes (over 100) to obtain sufficient information to solve the mathematical problem. These methods are also mainly used for research purposes, and are not necessarily suited to industrial applications, where calculations may have to be made in real time, and with an easy-to-use device.

The invention disclosed in U.S. Pat. No. 9,687,168 ([3]) consists of a method for surface electromyography of deep muscles. The invention consists in arranging a matrix of mono-polar electrodes encircling the circumference of a part of the body wherein the deep muscle to be investigated is included. The application example chosen concerns the measurement of the brachial muscle of the arm, using two arrays of 6 electrodes each, configured in two concentric circles around the arm. This method therefore requires 12 electrodes to characterize a muscle. Furthermore, this patent does not teach how to apply the deep muscle method to other anatomical regions, since a specific calibration phase of the muscle distribution of the investigated region is necessary for the subsequent application of the independent component analysis.

Finally, document WO2019097166 ([4]) relates to a device for measuring the specific muscular activity of one or more deep muscles of a subject's abdominal wall using surface EMG sensors, enabling the muscular activity of several deep muscles (denoted N) to be measured simultaneously, while obtaining a specific, that is, individualized, result for the activity of either one deep muscle, or several deep muscles at the same time, while being able to specifically attribute to each of the muscles the value of the muscular activity measured for that muscle. In this invention, the device does not allow specific, individualized measurement of the muscular activity of a set of muscles that also comprises superficial muscles. Moreover, the invention relates to the deep muscles of the abdominal wall only, without being transposable to any other anatomical site.

There are also methods based on the development and parameterization of signal classifiers.

Fajardo et al. (“EMG hand gesture classification using handcrafted and deep features”, Biomedical Signal Processing and Control, Volume 63, January 2021, 102210 ([5])) discloses a method using an MLP (multi-layer perceptron) classifier to classify EMG signals according to hand gesture. This method does not allow differentiation of the muscular activity of each muscle in the region, specifically and independently.

Arteaga et al. (“EMG-driven hand model based on the classification of individual finger movements”, Biomedical Signal Processing and Control, Volume 58, April 2020, 101834 ([6])) presents a method using an SVM (Support Vector Machines) classifier to classify EMG signals according to hand gesture and finger movements. This method does not allow differentiation of the muscular activity of each muscle in the region, specifically and independently.

There is therefore a real need for a method for measuring the specific muscular activity of a group of muscles comprising at least one deep muscle and at least one superficial muscle.

### DESCRIPTION OF THE INVENTION

The aim of the present invention is precisely to address these needs and disadvantages by providing a method for measuring the specific muscular activity of a group of muscles comprising at least one deep muscle and at least one superficial muscle.

Indeed, after considerable research, the Applicant has succeeded in developing a method for characterizing muscle co-contraction patterns in a given anatomical region, based solely on surface sensors, which can be non-invasive. Additionally, this muscle group contains at least one deep muscle and at least one superficial muscle.

Advantageously, the method of the invention makes it possible to take measurements on an anatomical site, then to optimize and parameterize a method for classifying the signals collected, thus obtaining a classifier. The classifier will then be able to classify any other signal, for example collected by non-invasive sensors such as surface EMGs, with a restricted number of sensors, into classes differentiated by the number and nature of muscles simultaneously contracted during the measurements, and the anatomical region covered by the measurement.

What is more, unlike the methods of the prior art, the method of the present invention allows the use of only a limited number of sensors once the classifier has been created, this number being less than the number of muscles to be characterized.

The field of the invention relates to a variety of applications such as functional rehabilitation, sporting performance, well-being and aesthetics, as well as prevention and functional diagnosis.

Thus, a first object of the invention relates to a computer-implemented method for classifying physiological signals arising from the specific muscular activity of a muscle group comprising at least one deep muscle and at least one superficial muscle of a subject, comprising the following steps:
a) acquiring electrical signals from at least one sensor placed on the skin of a subject,
b) pre-processing the electrical signals acquired during step a), to eliminate spurious noise and subject-motion artifacts,
c) extracting at least one time variable, at least one frequency variable, at least one time-frequency variable, at least one fractal variable, at least one cepstral variable and at least one statistical variable from the pre-processed signals from step b),
d) selecting variables by analyzing the variance to classify subsequent data according to selected classes, the selected variables forming a classifier,
e) implementing the classifier from the variables selected in step d),
f) evaluating classifier performance by cross-validation and performance indicators,
g) using the classifier to characterize the muscular activity of the muscle groups.

Advantageously, the method of the invention can be applied to any muscle or muscle group in the human or animal body. For example, the muscles of the abdominal wall, back, buttocks, arm, forearm, leg, thigh, face, neck, thorax, shoulder and foot.

For the purposes of this invention, the term “deep muscle” refers to any muscle lying beneath the visible outer muscle layers. These may for example be at least one muscle selected from the transverse abdominis, psoas, quadratus lumborum, oblique internus, ilio dorsalis, long dorsalis, intervertebral, supraspinatus, perineal muscles, multifidi, spinal muscles, in particular those connecting the spinous process and transverse processes of each vertebra, and the native musculature of the back.

For the purposes of this invention, the term “surface muscle” or “superficial muscle” refers to any muscle visible under the skin. This can be at least one muscle selected from the external oblique, rectus abdominis, scalene, sternocleidomastoid, trapezius, pectoralis major, deltoids, dorsalis major, intercostal muscles, biceps, triceps, forearm flexors or extensors, gluteals, abductors, adductors, hamstrings, quadriceps and gastrocnemius. Measurements can be collected prior to step a) on an anatomical region using at least one sensor, providing information on the contraction or relaxation of each muscle at any given moment, whether deep or superficial. Sensors can be positioned directly above one or more superficial muscles or deep muscles to be analyzed, or on both sides of a superficial muscle or deep muscle to be analyzed. Advantageously, for the method of the invention to be applicable, it is sufficient for the contraction of each muscle to be analyzed to be visible on the signal collected by the sensor(s) in the region, without this contribution alone being necessary to determine the state of contraction of said muscle.

The sensors used in step a) can be any surface sensor, that is, any device for sensing a physical phenomenon and outputting it as a signal, in this case an electrical signal (electrical signal acquisition step). These may be non-invasive sensors, for example to be placed and/or adhered on a subject's skin, or invasive sensors, for example to be introduced at least partially into the skin. Non-invasive sensors can for example include electromyographic (EMG) electrodes, patches, temporary electronic tattoos or medical imaging. Invasive sensors can for example be patches fitted with a needle that touches the muscle fiber directly. The sensors are those normally used for this type of measurement, and as such can be fitted with flexible conductors connecting them individually to recording means and computing means suitable for performing an independent component analysis. These may also be imaging devices commonly used for this type of measurement. Within the scope of the invention, invasive and non-invasive sensors can be used simultaneously, or one of the two types simultaneously. The number of sensors that can be used in step a) is unlimited.

Advantageously, the acquisition step a) of the method can be used to obtain a sufficiently large database of signals to be used as a training base, and to optimally parameterize the classifier.

Step b) of pre-processing the electrical signals can be any method known to the person skilled in the art for eliminating all or part of the spurious noise and subject-motion artifacts. This may involve signal filtering, for example by means of a high-pass filter with a cut-off frequency between 15 Hz and 50 Hz, in order to eliminate signal mean values and baseline variations. The signals can then be rectified using the absolute value of the signal.

In extraction step c), the variables can be chosen from those commonly used to analyze signals relating to muscular activity. Examples include signals commonly used to study EMG signals, such as:

Time indicators, such as:
Waveform Length (WL) (Cengiz Tepe, Mehmet Can Demir ([9]))
Average signal amplitude (MAV)
The sum of the amplitudes of the time signal divided by the maximum value of the signal (sum (amp)/max)
The temporal correlation between two measurement channels (corr), Hjorth parameters (Hj) such as activity (A), mobility or complexity (Jose Manuel Fajardo et al. ([10]); Noemi Gozzia et al. ([11]))
Skewness (or Sk) (José Manuel Fajardo et al. ([10]); Noemi Gozzia et al. ([11]); Firas Sabar Miften et al. ([12]))
Sequential indicators, such as:
Mean frequency (mf) (Cengiz Tepe, Mehmet Can Demir ([9]); Maria V. Arteaga et al. ([13]))
Median frequency (mdf) (Cengiz Tepe, Mehmet Can Demir ([9]); Maria V. Arteaga et al. ([13]))
Cutoff frequency (fp). This is the frequency value for which more than 60% of the power in the spectrum is prior to this value, with 60% being selectable between 50% and 95%.
Time-frequency indicators, such as:
Empirical mode decomposition (EMD) (Lingmei Ai et al. ([8])
immediate average frequency DWT (IAF_dwt) (Abdoulaye Thioune ([14]))

The database of indicators formed in step c) is also made up of indicators that are used in other technical fields, such as fractal analysis, which has been used for some years to analyze financial market signals or to model traffic on computer networks. Fractal variables that can be used include the following indicator:
Hurst exponent: H (Mario Cifrek et al. ([15]))

Another family of indicators which is not used in the prior art for analyzing EMG signals is cepstral analysis. Indeed, this method is generally used to process sound signals. In particular, it is used to determine voiceprints. At least one of the following indicators can be extracted from this type of analysis:
Cepstral coefficient (Ce) (Noemi Gozzia et al. ([11]))
calculations of temporal indicators such as mean, median, standard deviation (std) or root mean square (rms) on cepstral coefficients (Cci),
calculations of mathematical indicators such as minimum (min) and maximum (max) on cepstral coefficients (Cci)

The database formed in step c) also contains statistical indicators representing signal entropy-some commonly used in EMG, others not. These indicators can be chosen from:
sample entropy (or SampEn) (Noemi Gozzia et al. ([11])),
fuzzy entropy (or FuzzyEn) (16. Lorenz Kahl, Ulrich G. Hofmann ([16]))
Shannon entropy (ShanEn) (Arlene John et al. ([17]))

Advantageously, the variable values can then be put into matrix form, with each row representing a signal and each column corresponding to the variable calculated on that signal (indicator).

Advantageously, once the variables have been calculated on all the signals, the next step, that is, step d), can comprise selecting the set of indicators that will best enable future data to be classified according to the chosen classes, that is, according to criteria linked to the number and type of muscles contracted at the same time in the anatomical region covered by the measurement. This selection can be made using any method known to the person skilled in the art, for example by studying the individual variables calculated according to the indicators, in order to determine their discriminating power, alone and/or in combination, for all the muscles selected and the desired classes in the future classifier. Using their general knowledge, the person skilled in the art can select a combination of indicators based on the desired classes. For example, it is possible to choose an indicator with good discriminating power to separate two classes, in combination with an indicator with good discriminating power for two other classes, for example; in combination, they will then have very good discriminating power to separate the 3 classes, one by one. Different combinations of indicators can then be chosen and tested within the classifier to assess the combined performance of the chosen set.

This step comprises an Analysis of Variance (Anova). Advantageously, this analysis can be used to check whether the mean value of the variable is significantly different for the different classes. This analysis can also be used to estimate intra-group (that is, residual) and inter-group (that is, factorial) variances. Particularly advantageously, the factorial variance can be used to determine whether the values of the variable under study change based on the groups.

At the end of this selection step (step d)), a classifier can be generated by learning from the input data collected with the sensors disclosed above, which will be classified in a fair and known way, and from the selected variables The classifier is capable of correctly classifying the signals that will be measured on the non-invasive sensors that can be implemented from step g) onwards, and which are advantageously fewer in number than the number of muscles integrated into the group studied, based on the co-contraction patterns.

Advantageously, the subsequent step of evaluating classifier performance by cross-validation and performance indicators (step e)) can reduce analysis bias due to the size of the training base. In general, to evaluate the performance of a classification algorithm, the initial labeled base is divided into two subsets, called the training base and the test base, respectively, the first enabling the algorithm to be trained and the second being used to predict and evaluate these predictions. To obtain statistically significant results with this method, the initial base must be made up of a suitable number of signals, which the person skilled in the art can evaluate according to their knowledge of statistical tools. If this initial condition is not met, the cross-validation method can be used. The latter involves dividing the signal base into n subsets (n>2). These subsets must be of approximately the same size and made up of a homogeneous distribution of individuals representing the different classification groups. Once this distribution has been done, (n−1) subsets are used to form the algorithm's training base and one subset for the test base. Advantageously, this method makes it possible to predict the class of all the individuals in the initial database without using the same signals in the training and test bases.

Once cross-validation has been carried out, performance indicators such as sensitivity, specificity and ROC (Receiver Operating Characteristic) index can be calculated by any suitable means known to the person skilled in the art.

For example, a confusion matrix for classification algorithms can be constructed. This involves counting the number of true positives, false positives, true negatives and false negatives. From this confusion matrix, various performance indicators can be calculated, such as sensitivity, specificity and the ROC (Receiver Operating Characteristic) index. Sensitivity (Se) is the rate of true positives, while specificity (Spe) is the rate of false negatives. The quality of a model depends on the trade-off between sensitivity and specificity, which can be obtained by calculating the ROC (Receiver Operating Characteristic) index with the following equation:

ROC=(1−Spe)²+(1−Se)²

The indicator can be the rate of correct classification, which corresponds to the rate of correctly classified individuals.

Finally, step g) of using the classifier can be carried out, in particular by applying said classifier to new electrical signals from at least one “target” sensor placed on the skin of a subject. Advantageously, once the classifier has been optimally implemented (steps d and e), the information that can be gathered on the co-contraction of at least one superficial muscle and at least one deep muscle can be obtained only from non-invasive “target” sensors, which are fewer in number than the number of muscles studied in the group. Advantageously, the number of “target” sensors likely to be used once the classifier has been implemented can be strictly less than the number of muscles (or the sum of the number of deep muscles and superficial muscles) whose activity is to be characterized, while still being strictly greater than 0. In other words, if the aim is to characterize the activity of N muscles, the number of “target” sensors required to implement the invention is at most N−1. This can be a number between 1 and N−1, based on the number of muscles to be characterized. Advantageously, the “target” sensors are defined as soon as step a) is implemented, as they enable the classifier to be optimized and the right variables to be chosen. Of course, a number of “target” sensors equal to or greater than the number of muscles to be characterized can be used, but this is not a necessity to implement the invention and obtain its benefits.

Any method known to the person skilled in the art can be used for implementation. For example, this can be at least one method chosen from support vector machines (SVM) and multi-layer perceptron (MLP).

The SVM technique is a set of supervised training methods (that is, a method using a model and a training base) used to solve discrimination (that is, separation into homogeneous classes) and regression problems. This technique is often used as a linear classifier. The principle is as follows: a function h(x), which is a linear combination, is called the kernel. It takes an individual as input and gives a class as output. For this function to be able to determine an individual's class, it first learns to differentiate between them by means of a training base. The training base contains a set of individuals whose class is known a priori. These individuals have characteristics described by the function h(x). The separation between the different groups is a hyperplane, located at h(x)=0. If we take two classes, for example, all points with h(x)>0, then they are in class 1; otherwise they are placed in class two. Once the function has learned to discriminate between the different groups, a new individual is provided as input, after which it returns its group.

The multilayer perceptron (MLP) is a kind of artificial neural network. The MLP can be made up of several layers of neurons, each layer having a variable number of neurons. All the neurons in one layer are connected to neurons in adjacent layers. The link between two neurons is weighted by a coefficient. This method requires training. To train the neural network, it is given the characteristics of an individual whose class is known, and these characteristics are then passed through each layer of the perceptron. Once the result has been retrieved, the MLP calculates the error between the true class and the predicted class, often using a root mean square. If the error is large, then the neuron weights are modified; otherwise, it moves on to the next sample. This method adjusts the neural network until a good predictor is obtained. Once training is complete, the perceptron is given new samples as input, and it calculates their classes.

A further object of the invention relates to a computer program product downloadable from a communication network and/or stored on a computer-readable medium and/or executable by a microprocessor, characterized in that it comprises program code instructions for executing the computer-implemented method of classifying physiological signals, when executed on a computer.

Another object of the invention relates to a method for measuring the specific muscular activity of a muscle group comprising at least one deep muscle and at least one superficial muscle of a subject, comprising the following steps:
1) acquiring electrical signals from at least one sensor placed on the skin of a subject,
2) pre-processing the electrical signals acquired during step (1), to eliminate spurious noise and subject-motion artifacts,
3) extracting the variables which have been selected during the development of the classifier as previously disclosed (steps a) to g) of the computer-implemented method of classifying physiological signals derived from the specific muscular activity of a muscle group comprising at least one deep muscle and at least one superficial muscle of a subject 4) classifying the variables extracted in step 3) by means of the classifier as previously defined.

Advantageously, this method makes it possible to characterize a muscular co-contraction pattern of at least one deep muscle and at least one superficial muscle simultaneously.

The definitions of terms given for the computer-implemented method of classifying physiological signals can be transposed to the measuring method.

Since the methods of the invention are computer-implemented, they may involve any computer device that can implement the various steps, and perform other routine software functions such as data recording, data archiving, display, for example by means of a computer monitor that can display electromyographic waveforms.

Another object of the invention relates to the use of the measurement method of the invention, for an application selected from functional rehabilitation, muscle strengthening for well-being and/or aesthetic purposes, prevention of lumbar, spinal, sports or pelvic pathologies, and functional diagnosis.

Other advantages may be seen by the person skilled in the art by reading the following examples, shown by the appended figures provided by way of illustration.

### BRIEF DESCRIPTION OF THE FIGURES

FIGS. 1A-1B show initial measurements taken at 4 measurement points via surface EMG sensors (1a, 1b, 1c, 1d, 1e, 1f, 1g, 1h) for future classifier use on target sensors “TR1” (1e, 1f) and “TR2” (1g, 1h) (EO=External Oblique; RA=Rectus Abdominis; TR=Transverse).

FIGS. 2A-2C show three measurements taken with surface EMG sensors (1a, 1b, 1c, 1d, 1e, 1f, 1g, 1h for FIG. 2A, 1a, 1b, 1g, 1h, 1i, 1j, 1k, 11 for FIGS. 2B and 1e, 1f for FIG. 2C) and only for the measurement dedicated to classifier parameterization and training base creation. This embodiment was used for a classifier optimized with target sensors TR1 and TR2, and for another classifier optimized with target sensors L1 and L2.

FIGS. 3A-3B show an initial signal (FIG. 3A) and a pre-processed signal (FIG. 3B).

FIGS. 4A-4F show the distribution of the “rms” variable for all groups (FIG. 4A), the distribution of the “Complexity” variable for all groups (FIG. 4B), the distribution of the “mf” variable for all groups (FIG. 4C), the distribution of the “median” variable for all groups (FIG. 4D), the distribution of the “mdf” variable for all groups (FIG. 4E), the distribution of the “H” variable for all groups (FIG. 4F).

FIGS. 5A-5B show the correct classification rates for 3-variable classifiers: [rms, Complexity, mf] (FIG. 5A) and 4-variable classifiers: [median, mf, mdf, H] (FIG. 5B).

FIGS. 6A-6P show the distribution of the “mean” variable for all groups (FIG. 6A), the distribution of the “std” variable for all groups (FIG. 6B), the distribution of the “median” variable for all groups (FIG. 6C), the distribution of the “min” variable for all groups (FIG. 6D), the distribution of the “rms” variable for all groups (FIG. 6E), the distribution of the “max” variable (FIG. 6F), the distribution of the “sum (amp)/max” variable for all groups (FIG. 6G), the distribution of the “WL” variable for all groups (FIG. 6H), the distribution of the “MAV” variable for all groups (FIG. 6I), the distribution of the “Complexity” variable for all groups (FIG. 6J), the distribution of the “mf” variable for all groups (FIG. 6K), the distribution of the “mdf” variable for all groups (FIG. 6L), the distribution of the “corr” variable for all groups (FIG. 6M), the distribution of the “f(p)” variable for all groups (FIG. 6N), the distribution of the “SampEn” variable for all groups (FIG. 6O), the distribution of the “H” variable (FIG. 6P).

FIG. 7 shows the rate of correct classification following the selection step disclosed in example 4.

FIGS. 8A-8F show the distribution of the “mf” variable for all groups (FIG. 8A), the distribution of the “mdf” variable for all groups (FIG. 8B), the distribution of the “fp” variable for all groups (FIG. 8C), the distribution of the “corr” variable for all groups (FIG. 8D), the distribution of the “wl” variable for all groups (FIG. 8E), the distribution of the “rms” variable for all groups (FIG. 8F).

FIGS. 9A-9B show the results obtained and the correct classification rate calculated using the method disclosed in example 5.

### EXAMPLES

### Example 1: Example of how to Collect Physiological Measurements

Measurements are collected over an anatomical region comprising N muscles, including at least one deep muscle, using multiple surface EMG sensors (1a, 1b, 1c, 1d, 1e, 1f, 1g, 1h) to provide information on the contraction or relaxation of each muscle at any given moment. These include so-called “target” sensors, which are non-invasive surface EMG sensors, of which there are a maximum of N−1. These sensors measure the signals to be classified by the classifier developed by the method.

The data collection makes it possible to obtain a sufficiently large database of signals to be used as a training base, and to optimally parameterize the classifier.

4 data sets were used. For these data, signals are collected either by surface EMG sensors or by ultrasound imaging. The chosen anatomical region is the abdominal wall, with targeted muscles including the transverse abdominis, internal obliques, external obliques and rectus abdominis muscles.

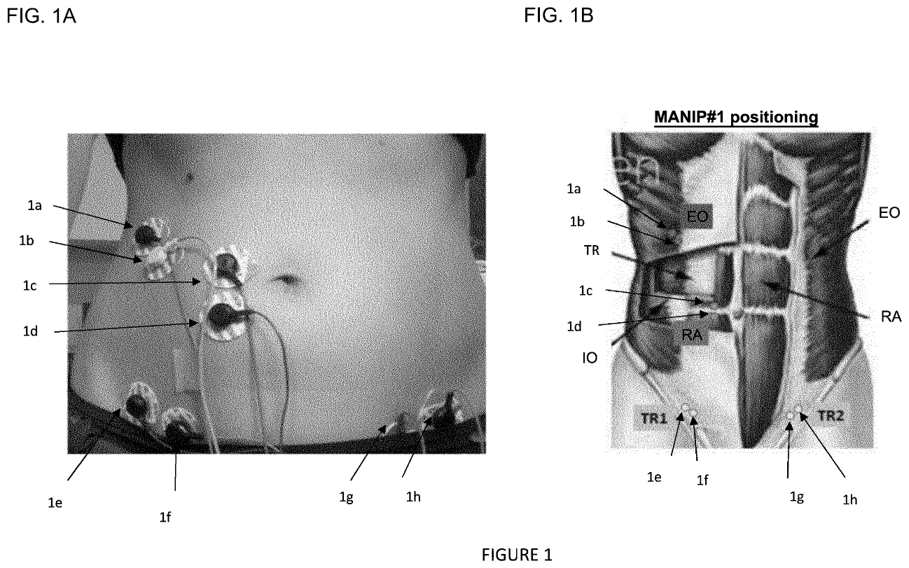

Dataset #1: initial measurements with 4 surface EMG measurement points (1a, 1b, 1c, 1d, 1e, 1f, 1g, 1h) for future classifier use on target sensors “TR1” (1e, 1f) and “TR2” (1g, 1h) as shown in FIG. 1 (EO=external oblique; RA=rectus abdominis; TR=transverse).

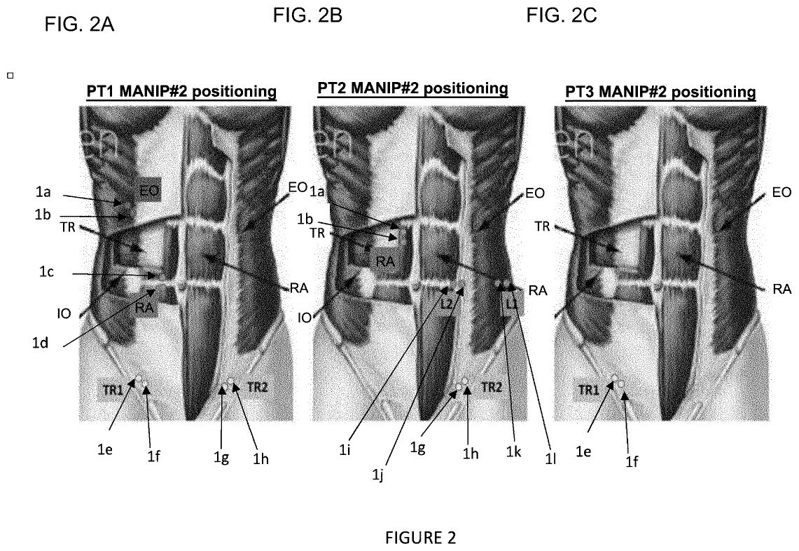

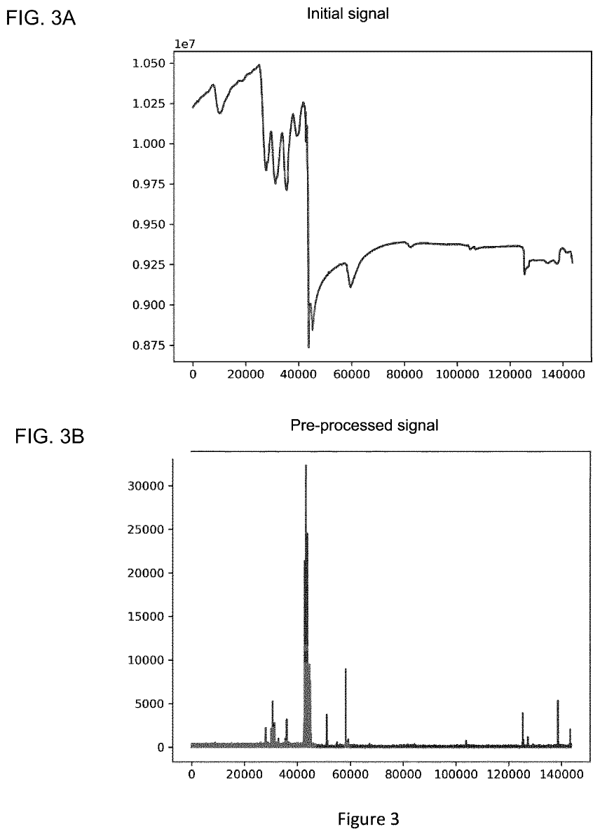

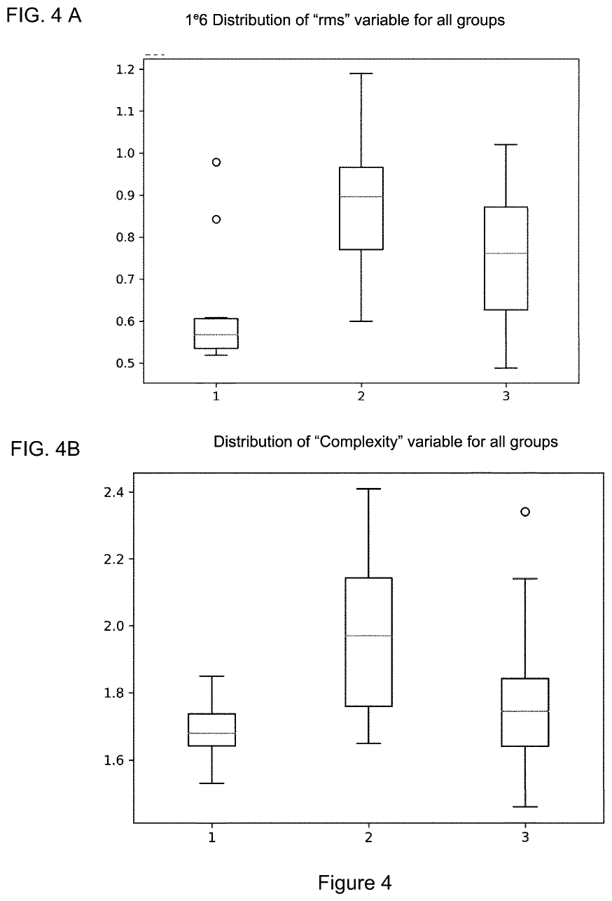

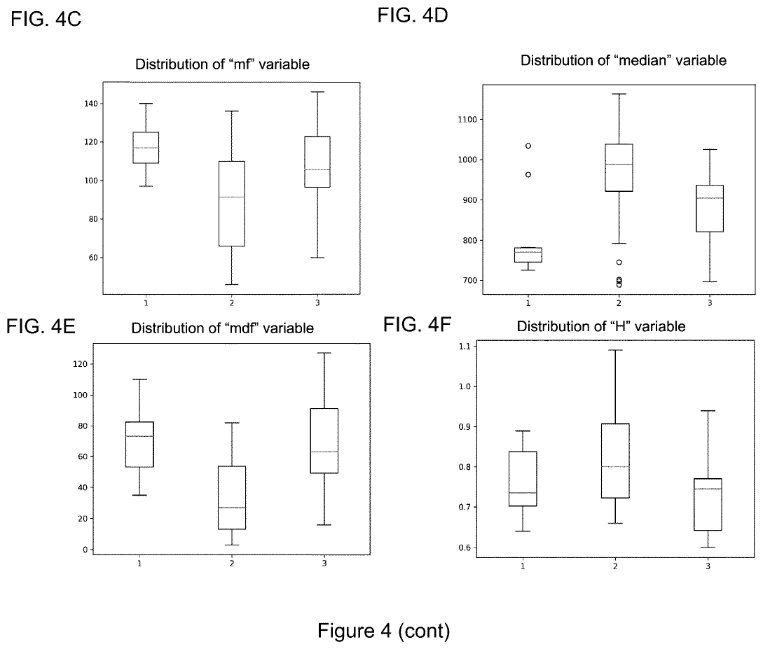

Dataset #2: again based on surface EMG sensors only, for measurements dedicated to classifier parameterization and training base creation. Three different positions are tested, as shown in FIGS. 2A-2C. The so-called target sensors that can subsequently be used as inputs to the classifier parameterized by the method of the invention can be the “TR1” and “TR2”, or the “L1”, or the “L2”.

Dataset #3: Initial measurements are again acquired with surface EMG sensors, placed at the following locations: 2 sensors on REO (right external oblique), 2 sensors on LEO (left external oblique), 2 sensors on URA (upper rectus abdominis), 2 sensors on LRA (lower rectus abdominis), 2 sensors on RIO (right internal oblique), 2 sensors on LIO (left internal oblique), 2 sensors on ILAT (internal lateral), 2 sensors on ELAT (external lateral), on a first subject; 2 sensors on REO, 2 sensors on LEO, 2 sensors on LRA, 2 sensors on RIO, 2 sensors on LIO, 2 sensors on ILAT, 2 sensors on ELAT, 2 sensors on GM (gluteus medius) on a second subject.

Dataset #4: This time, data is measured using surface EMG sensors and ultrasound video. Ultrasound imaging measurements are taken via a linear probe positioned according to the Université de Montréal protocol to image the abdominal region. The images allow real-time measurement of contractions of the internal and external obliques and the transverse abdominis.

### Example 2: Building a Classifier

Preliminary work consisted in:
exploratory analyses of EMG signals collected by surface EMG sensors placed in the lower abdominal region, and thus containing information related to contractions of the various muscles “visible” by the electrodes, namely at a minimum, the rectus abdominis, the transverse abdominal muscle and the internal obliques. The exploratory analyses consisted in analyzing the time-frequency characteristics of the signals as a function of the contraction patterns of these three muscles. The following were implemented and analyzed:
periodograms
Empirical Mode Decomposition (EMD),
Choi-Williams distributions
wavelet transforms
These analyses enabled identification of indicators that could be good candidates for classifying signals according to responses corresponding to moments when the transverse abdominal muscle is contracting and moments when the transverse abdominal muscle is not contracting.
Finally, several classifiers were implemented based on methods found in the scientific literature. The candidate indicators identified above were parameterized as inputs to these classifiers in order to test them and measure their theoretical effectiveness (by cross-validation on known data).

Two types of classifiers were considered during this exploratory research, firstly SVM (Support Vector Machine) type classifiers, then MLP (MultiLayer Perceptron) type classifiers (Demont A, Lemarinel M.: “Échographie musculaire de l'abdomen: principes de base et applications cliniques pour la lombalgie commune chronique” [Muscular Sonogram of the Abdomen: Basic Principles and Clinical Applications for Chronic Common Low Back Pain]. Kinesither Rev. 2017; 17(182): 41-49 ([7]).

For SVM classifiers, different input variables were used, and different kernels were tested to see which gave the best specificity.

The same analytical method was applied to MLP classifiers, also varying the number of neurons per layer. For the input variables, the singular values extracted from the empirical mode decomposition were first used (following a method proposed by Lingmei Ai, Jue Wang, Ruoxia Yao (“Classification of parkinsonian and essential tremor using empirical mode decomposition and support vector machine” ([8])).

Following this, the classifiers were fed with other simple spectral and temporal indicators, such as average frequency or temporal correlation between the two channels.

In all, almost 511 different configurations were tested on these classifiers, and classification efficiencies of between 70% and 80% were achieved.

The method was then optimized to be usable on any dataset collected and labeled by surface EMG measurements. “Labeled” means able to indicate which muscle(s) are contracted or relaxed at any given moment and on any given measurement channel. The method was then used to parameterize classifiers achieving classification efficiencies of the order of 96% over the abdominal wall region.

The inventors have succeeded in defining indicators other than those used in the prior art, enabling higher classification efficiencies to be achieved.

Indeed, the methods used in the prior art to develop EMG signal classifiers often consist in taking a “homogeneous” set of indicators, that is, the indicators will all be of the same type: either the classifier is based on temporal indicators, or on time-frequency indicators, or on more exotic indicators, but only these. In the present invention, the results have been greatly optimized and improved by choosing a set of hybrid indicators, some from temporal analysis, others from time-frequency analysis, or from fractal analysis.

### Example 3: Example of Classifier Implementation

The abdominal wall is a multi-layered compartment. It consists of:
a layer of skin and a layer of subcutaneous tissue,
superficial fascia and a deep fascia,
a muscular layer,
the fascia transversalis and the peritoneum, which protects the viscera.

The muscular layer of the abdominal wall is made up of 5 even muscles: two vertical muscles, the rectus abdominis and the pyramidalis, and three lateral muscles, the external oblique, the internal oblique and the transversus abdominis. The main role of these muscles is to protect the viscera. They also generate and regulate intra-abdominal pressure.

The sensors used to measure the data are labeled BBP (under anterior superior iliac spine). They pick up signals from the following muscles: rectus abdominis, external oblique, internal oblique, transversus abdominis, psoas and perineum.

Other signals were also acquired, such as signals from the rectus abdominis using surface electrodes placed according to SENIAM guidelines (Surface ElectroMyoGraphy for the Non-Invasive Assessment of Muscles). Ultrasound videos were also acquired to improve expert classification.

The method is followed to develop a classifier that can discriminate three classes among signals measured by two EMG surface electrodes, positioned on “BBP” locations (under the anterior superior iliac spine), which therefore do not correspond to any SENIAM-referenced positioning. Based on this placement, the method can discriminate between the transverse abdominus, the internal obliques and the rectus abdominis.

For step 5 (Performance evaluation by cross-validation and performance indicators), the classifier used is an SVM with a linear kernel. The indicator base used at the start of this analysis was composed of the following indicators: rms, Hj, mf, mdf, median, H, WL, Sk, M, FuzzyEn

Following step 4 (Variable selection), two sets of indicators were selected for implementation:
Set 1: rms, Hj and mf
Set 2: median, mf, mdf, H

Cross-validation is used to evaluate the performance of the classifiers, by splitting the initial training base into 5 subgroups

| | True | | |
|---|---|---|---|
| Predicted | 1 | 2 | 3 |
| 1 | 9 | 1 | 0 |
| 2 | 0 | 8 | 2 |
| 3 | 0 | 2 | 8 |

Confusion matrix of [‘ms’, ‘Hj’, ‘mf’]

| | True | | |
|---|---|---|---|
| Predicted | 1 | 2 | 3 |
| 1 | 2 | 8 | 0 |
| 2 | 1 | 6 | 3 |
| 3 | 0 | 1 | 9 |

Confusion matrix of [‘median’, ‘mf’, ‘mdf’, ‘H’]

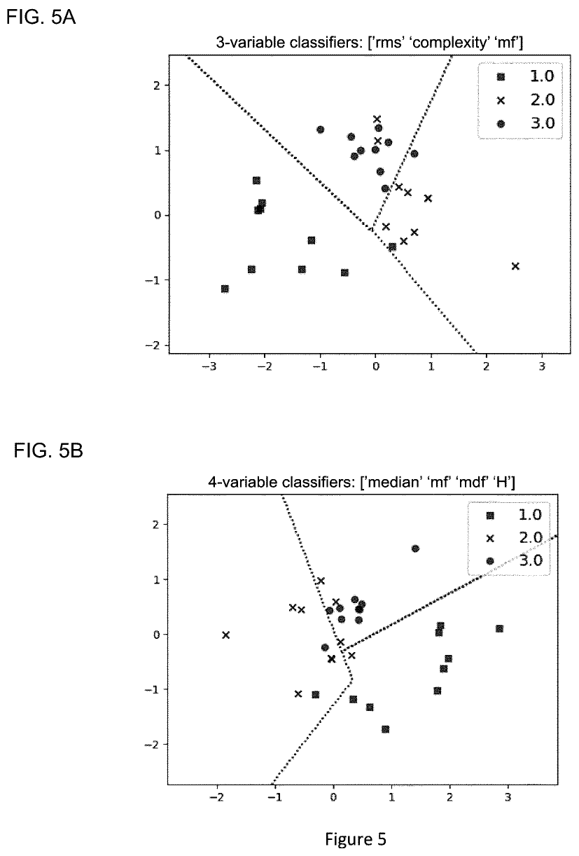

We obtained the classifiers and correct classification rates shown in FIGS. 5A-5B.

### Example 4: Example of Classifier Implementation

The signals used in this example are the same as in the previous example. The classifiers implemented here seek to discriminate between specific groups of contracted muscles. Five classes are highlighted:
1=TRA.
2=TRA & RA
3=TRA & RA & IO.
4=RA.
5 IO

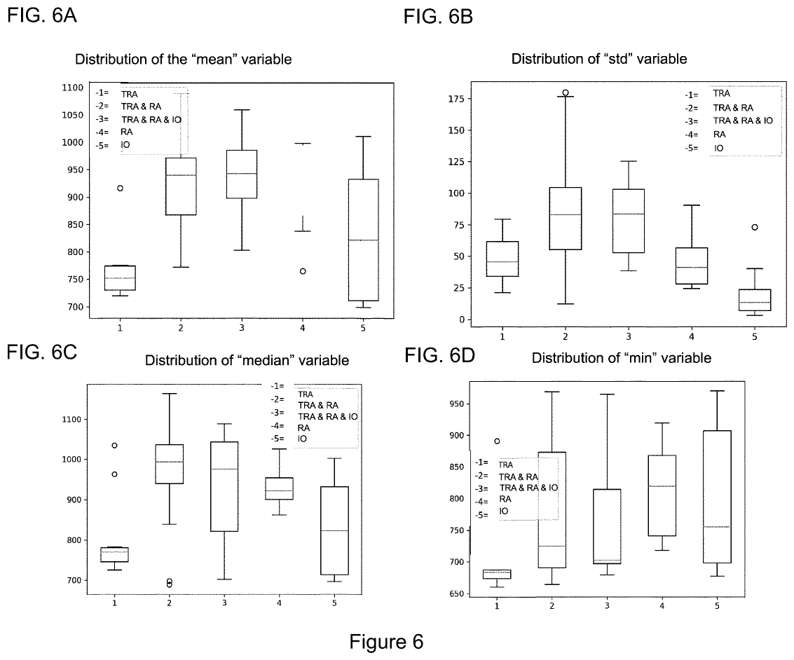

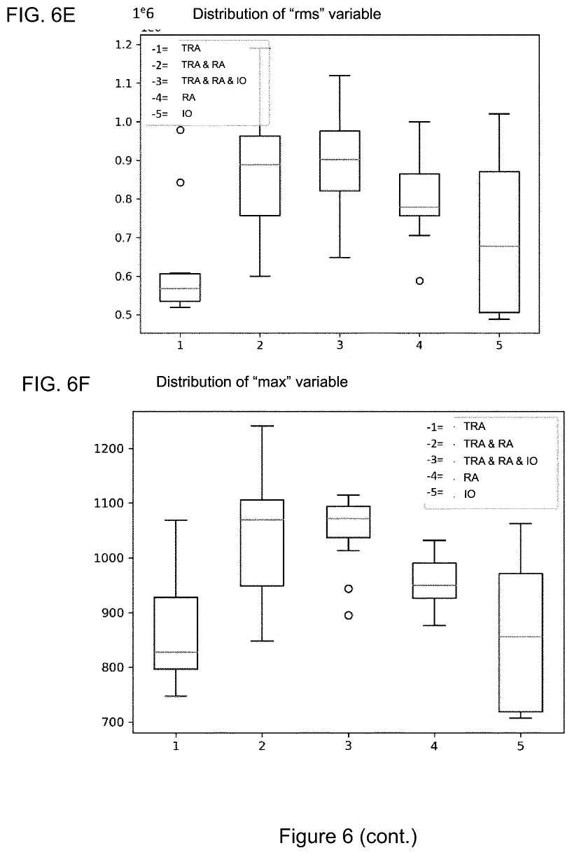

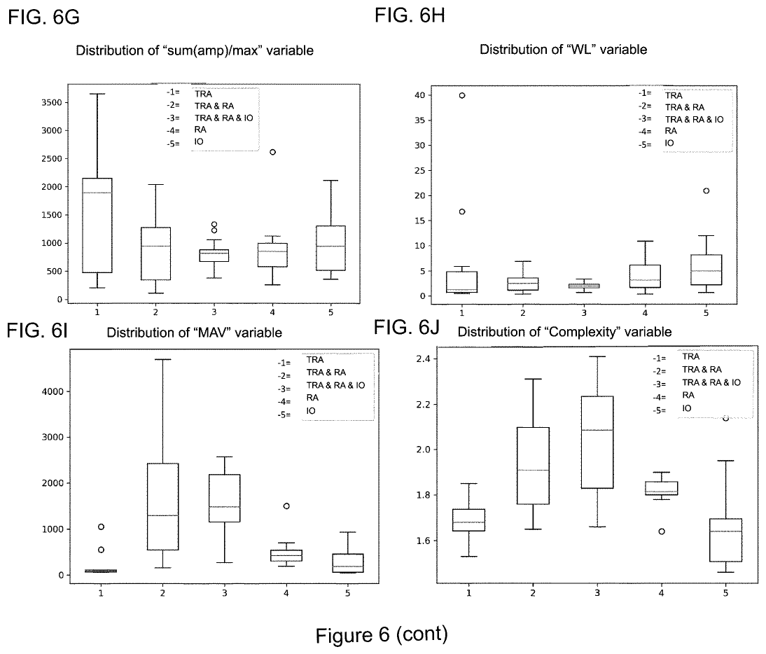

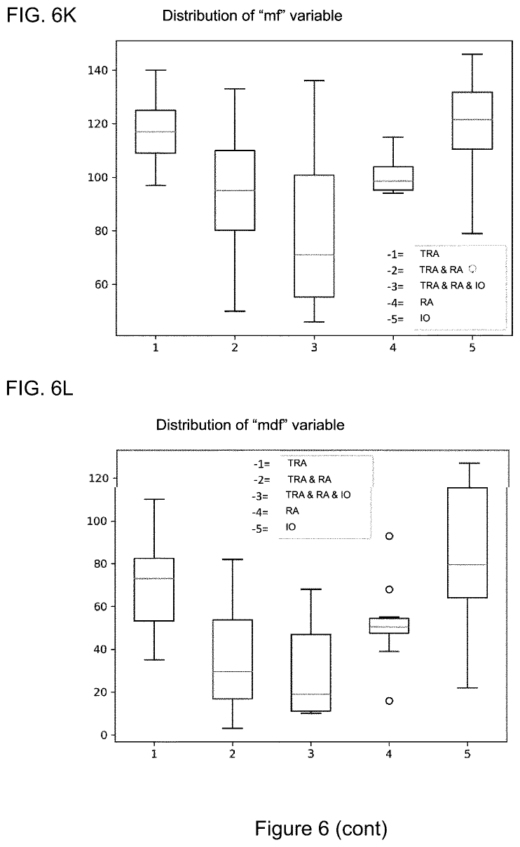

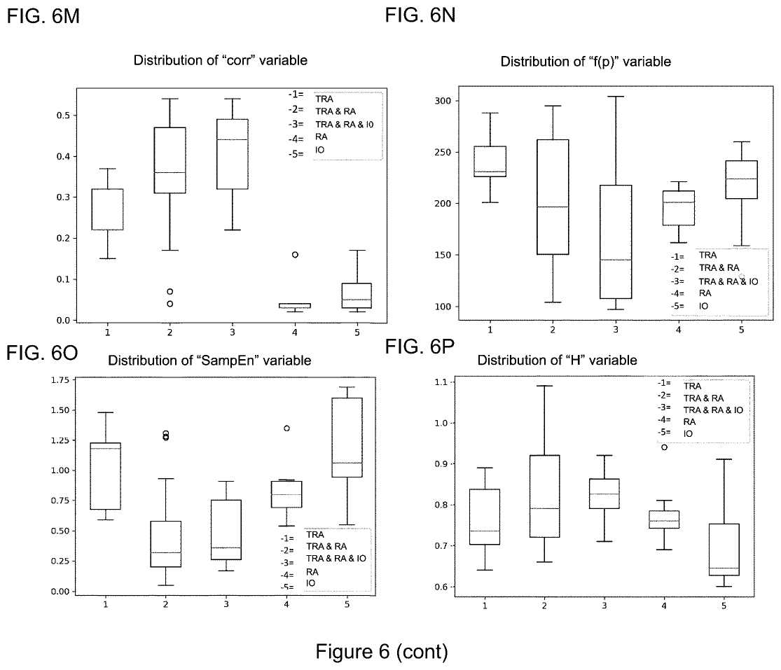

The following indicators were chosen to form the training base: mean, std, median, min, rms, max, sum (amp)/max, wl, MAV, Hj (Complexity), mf, mdf, corr, f(p) and sampEn. FIGS. 6A-6P shows the distribution of each selected indicator according to classification group.

Following a selection stage, the following dataset was taken as the training base:

Set 1: ‘mean’ ‘std’ ‘median’ ‘min’ ‘rms’ ‘max’ ‘MAV ‘Complexity’ ‘mdf’ ‘corr’ ‘f(p)’ ‘SampEn’ ‘H’

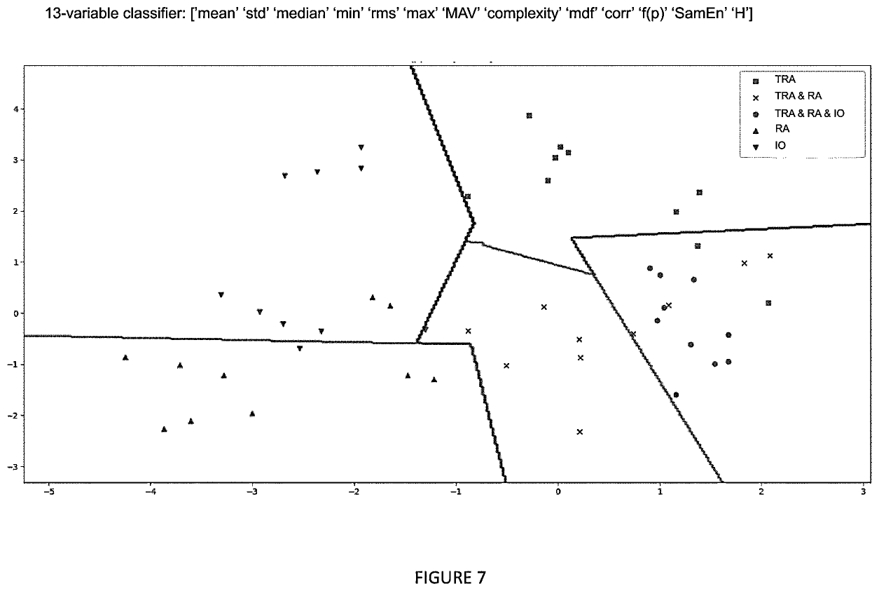

Correct classification rates are shown in FIG. 7.

| | 1 | 2 | 3 | 4 | 5 |
|---|---|---|---|---|---|
| 1 | 8 | 0 | 2 | 0 | 0 |
| 2 | 0 | 5 | 4 | 1 | 0 |
| 3 | 0 | 2 | 8 | 0 | 0 |
| 4 | 0 | 0 | 0 | 8 | 2 |
| 5 | 0 | 0 | 0 | 5 | 5 |

Confusion matrix of [‘mean’ ‘std’ ‘median’ ‘min’ ‘rms’ ‘max’ ‘MAV ‘Complexity’ ‘mdf’ ‘corr’ ‘f(p)’ ‘SampEn’ ‘H’]

### Example 5: Example of Classifier Implementation

For this third example of classifier implementation, the signals used are taken from the data collection shown in FIGS. 2A-2C. The studied region is still the abdominal cavity. However, the placement of the “target” electrodes differs from the two previous examples. Indeed, the so-called target sensors for the future use of the classifier here are the L1 and L2 sensors.

Three classes are highlighted:
1=TRA
2=TRA & RA
3=RA

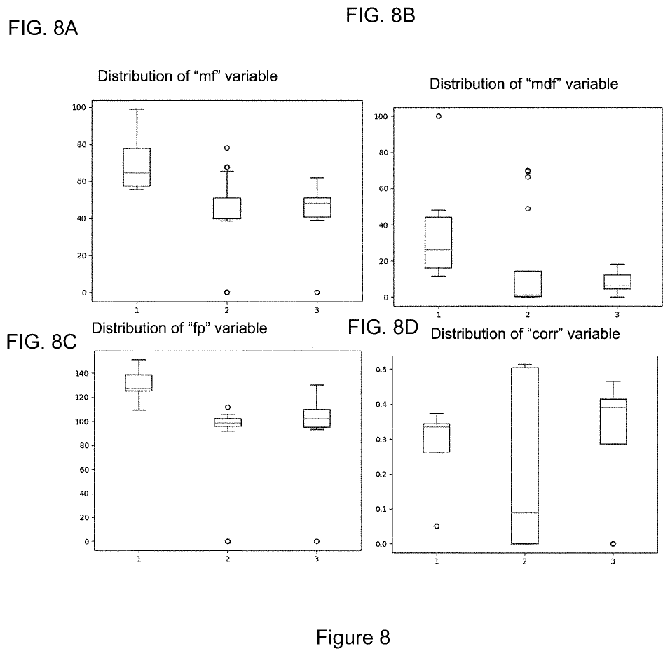

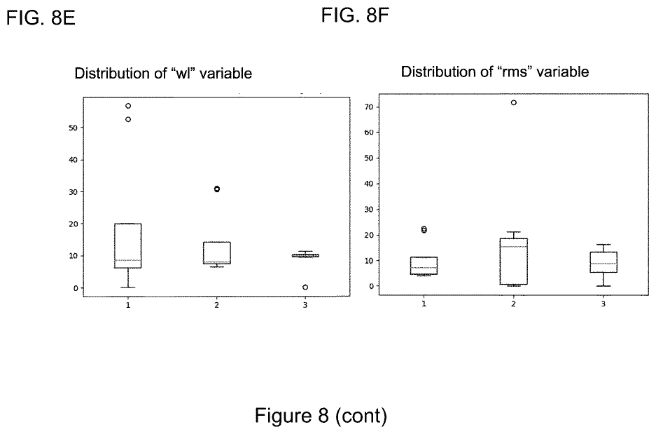

The following indicators were chosen to form the training base: sum (amp)/max, wl, Activity, Hj, Skewness, AUC/max, mf, mdf, corr and f(p). FIGS. 8A-8F shows the distribution of selected indicators according to classification groups.

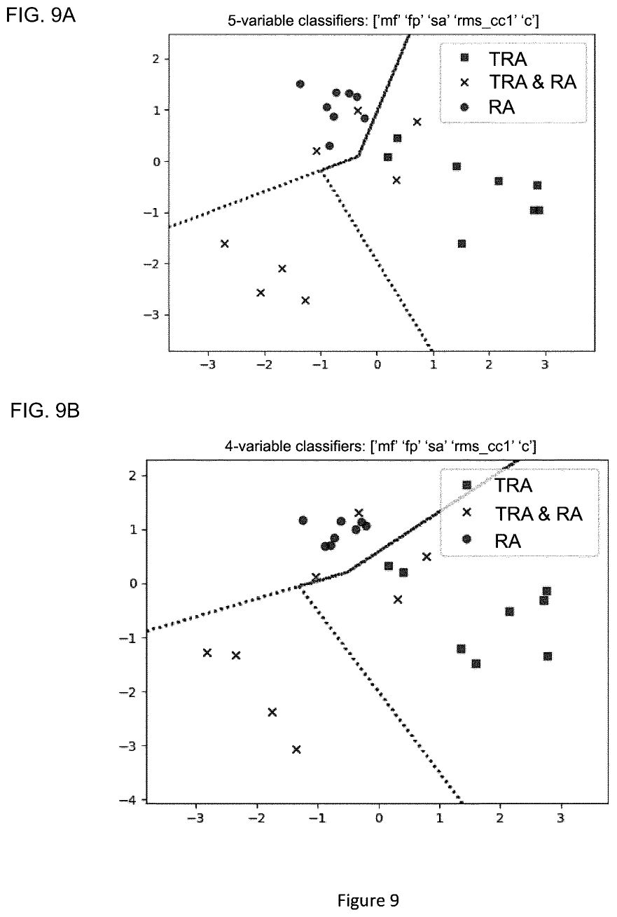

FIGS. 9A-9B shows the results obtained and the calculated rate of correct classification.

### LISTS OF REFERENCES

1. McGill et al. (1996, J. Biomechanics, Vol. 29, No. 11, pp. 1503-1507.
2. Jesinger and Stonick (1994, 6th IEEE DSP Workshop Proc., pp. 57-60.
3. U.S. Pat. No. 9,687,168.
4. WO2019097166.
5. Fajardo et al.: “EMG hand gesture classification using handcrafted and deep features”, Biomedical Signal Processing and Control, Volume 63, January 2021, 102210.
6. Arteaga et al.: “EMG-driven hand model based on the classification of individual finger movements”, Biomedical Signal Processing and Control, Volume 58, April 2020, 101834.
7. Demont A, Lemarinel M.: “Échographie musculaire de l'abdomen: principes de base et applications cliniques pour la lombalgie commune chronique”. Kinesither Rev. 2017; 17 (182): 41-49.
8. Lingmei Ai et al. (“Classification of parkinsonian and essential tremor using empirical mode decomposition and support vector machine”. Digital Signal Processing; Volume 21, Issue 4, July 2011, Pages 543-550.
9. Cengiz Tepe, Mehmet Can Demir. The effects of the number of channels and gyroscopic data on the classification performance in EMG data acquired by Myo armband. Journal of Computational Science; Volume 51, April 2021, 101348.
10. Jose Manuel Fajardo, Orlando Gomez, Flavio Prieto: “EMG hand gesture classification using handcrafted and deep features. Biomedical Signal Processing and Control; Volume 63, January 2021, 102210.
11. Noemi Gozzia, Lorenzo Malandri, Fabio Mercorio, Alessandra Pedrocchi: “XAI for myo-controlled prosthesis: Explaining EMG data for hand gesture classification”. Knowledge-Based Systems; Volume 240, 15 Mar. 2022, 108053.
12. Firas Sabar Miften, Mohammed Diykh, Shahab Abdulla, Siuly Siuly, Jonathan H. Green, Ravinesh C. Deo: “A new framework for classification of multi-category hand grasps using EMG signals”. Artificial Intelligence in Medicine; Volume 112, February 2021, 102005.
13. Maria V. Arteaga, Jenny C. Castiblanco, Ivan F. Mondragon, Julian D. Colorado, Catalina Alvarado-Rojas: “EMG-driven hand model based on the classification of individual finger movements”. Biomedical Signal Processing and Control; Volume 58, April 2020, 101834.
14. Abdoulaye Thioune: “Décomposition modale empirique et décomposition spectrale intrinsèque: applications en traitement du signal et de l'image” [Empirical modal decomposition and intrinsic spectral decomposition: applications in signal and image processing]. Thesis defended on Sep. 27, 2016. HAL Id: tel-01372335.
15. Mario Cifrek, Vladimir Medved, Stanko Tonkovic, Sasa Ostoji: “Surface EMG based muscle fatigue evaluation in biomechanics”. Clinical Biomechanics; Volume 24, Issue 4, May 2009, Pages 327-340.
16. Lorenz Kahl, Ulrich G. Hofmann: “Comparison of algorithms to quantify muscle fatigue in upper limb muscles based on sEMG signals”. Med Eng Phys; 2016 November; 38 (11): 1260-1269.
17. Arlene John, Aravind E. Vijayan, A. P. Sudheer: “Electromyography based control of robotic arm using entropy and zero crossing rate”. AIR '15: Proceedings of the 2015 Conference on Advances In Robotics July 2015 Article No.: 69 Pages 1-6.

1. A computer-implemented method for classifying physiological signals arising from the specific muscular activity of a muscle group comprising at least one deep muscle and at least one superficial muscle of a subject, comprising the following steps:
a) acquiring electrical signals from at least one sensor placed on the skin of a subject,
b) pre-processing the electrical signals acquired during step a), to eliminate spurious noise and subject-motion artifacts,
c) extracting at least one time variable, at least one frequency variable, at least one time-frequency variable, at least one fractal variable, at least one cepstral variable and at least one statistical variable from the pre-processed signals arising from step b),
d) selecting variables by analyzing the variance to classify subsequent data according to selected classes, the selected variables forming a classifier,
e) implementing the classifier from the variables selected in step d),
f) evaluating classifier performance by cross-validation and performance indicators,
g) using the classifier to characterize the muscular activity of the muscle groups.

2. The method according to claim 1, wherein the at least one sensor is selected from non-invasive sensors such as electromyographic (EMG) or medical imaging sensors, patches or temporary electronic tattoos and invasive sensors, such as patches with a needle.

3. The method according to claim 1, wherein the number of sensors used in step a. is strictly less than the sum of the number of deep muscles and superficial muscles, while still being strictly greater than 0.

4. The method according to claim 1, wherein the at least one fractal variable is the Hurst exponent.

5. The method according to claim 1, wherein the at least one cepstral variable is chosen from cepstral coefficients (Cci), calculations of temporal indicators such as mean, median, standard deviation (std) or root mean square (rms) on the cepstral coefficients (Cci), and calculations of mathematical indicators such as minimum (min) and maximum (max) on the cepstral coefficients (Cci).

6. The method according to claim 1, wherein the at least one statistical variable is chosen from sample entropy (SampEn), fuzzy entropy (FuzzyEn) and Shannon entropy (ShanEn).

7. The method according to claim 1, wherein:
the at least one temporal variable is selected from the length of the waveform (WL), the average signal amplitude (MAV), the sum of the temporal signal amplitudes divided by the maximum signal value (sum (amp)/max), the temporal correlation between two measurement channels (corr), the Hjorth parameters (Hj) such as activity (A), mobility or complexity and skewness (Sk),
the at least one frequency variable is selected from the mean frequency (mf), the median frequency (mdf) and the cut-off frequency (fp), and
the at least one time-frequency variable is chosen from Empirical Mode Decomposition (EMD) and Immediate Average Frequency DWT (IAF_dwt).

8. The method according to claim 1, wherein the variance analysis comprises intra-group variance analysis and/or inter-group variance analysis.

9. The method according to claim 1, wherein the implementation is carried out by at least one method chosen from support vector machines and the multilayer perceptron.

10. The method according to claim 1, wherein said at least one deep muscle and said at least one superficial muscle is a muscle of the abdominal wall, a dorsal muscle, a gluteal muscle, an arm muscle, a forearm muscle, a leg muscle, a thigh muscle, a face muscle, a neck muscle, a thorax muscle, a shoulder muscle, a foot muscle.

11. The method according to claim 1, wherein said at least one deep muscle is selected from the transverse abdominis, psoas, quadratus lumborum, oblique internus, ilio dorsalis, long dorsalis, intervertebral, supraspinatus, perineal muscles, multifidi, spinal muscles, in particular those connecting the spinous process and transverse processes of each vertebra, and the native musculature of the back.

12. The method according to claim 1, wherein said at least one superficial muscle is selected from the external oblique, rectus abdominis, scalene, sternocleidomastoid, trapezius, pectoralis major, deltoids, dorsalis major, intercostal muscles, biceps, triceps, forearm flexors or extensors, gluteals, abductors, adductors, hamstrings, quadriceps and gastrocnemius.

13. A method for measuring the specific muscular activity of a muscle group comprising at least one deep muscle and at least one superficial muscle of a subject, comprising the following steps:
1) acquiring electrical signals from at least one sensor placed on the skin of a subject,
2) pre-processing the electrical signals acquired during step (a), to eliminate spurious noise and subject-motion artifacts,
3) extracting at least one time variable, at least one frequency variable, at least one time-frequency variable, at least one fractal variable, at least one cepstral variable and at least one statistical variable from the pre-processed signals arising from step (b),
4) classifying the variables extracted in step 3) using the classifier as defined in claim 12.

14. A use of a measuring method according to claim 13, for an application selected from functional rehabilitation, muscle strengthening for well-being and/or aesthetic purposes, prevention of lumbar, spinal, sports or pelvic pathologies, and functional diagnosis.

15. A computer program product downloadable from a communication network and/or stored on a computer-readable medium and/or executable by a microprocessor, characterized in that it comprises program code instructions for executing the method according to claim 1, when it is executed on a computer.
